SEQUENCE LISTING (1 sequence)

SEQ ID NO: 1 - DNA, 25 bp, synthetic construct
  source 1..25: mol_type = other DNA; note = 9C-PC
  misc_feature 8^9: note = photocleavable aminotag phosphoramidite group
acttacctcccccccccaggtaagt

(19) United States
(12) Patent Application Publication
(10) Pub. No.: US 2026/0284193 A1
(43) Pub. Date: Sep. 24, 2026

(54) CYTOTOXIC AGENT

(71) Applicant: UNIVERSITEIT LEIDEN, Leiden (NL)

(72) Inventors: Donny DE BRUIN, Leiden (NL); Dirk BOUWMEESTER, Leiden (NL); Doris Marianne HEINRICH, Leiden (NL)

(21) Appl. No.: 19/477,738

(22) PCT Filed: Apr. 23, 2024
(86) PCT No.: PCT/NL2024/050210

§ 371 Date: Oct. 22, 2025

(30) Foreign Application Priority Data
Apr. 24, 2023 (NL) 2034670

(51) Int. Cl. A61K 41/00; A61K 33/38; A61P 35/00

(52) U.S. Cl. CPC A61K 41/0042; A61K 33/38; A61P 35/00

(57) ABSTRACT

Provided is a cytotoxic agent which is activatable by exposure to light. The agent comprises a metal cluster and a molecule comprising a photocleavable group. The molecule forms a structure which at least partially encloses the metal cluster so as to prevent the metal cluster from interacting with a surrounding environment and wherein, upon exposure to light, cleavage of the photocleavable group changes the structure of the molecule such that the metal cluster is able to interact with the surrounding environment.

### FIELD OF THE INVENTION

The present invention relates to a cytotoxic agent which is activatable by exposure to light. More particularly, the invention relates to an agent comprising a metal cluster which is at least partially enclosed by a molecule comprising a photocleavable group, wherein upon exposure to light cleavage of the photocleavable group alters the structure of the molecule such that the metal cluster is able to interact with the surrounding environment. Also provided are formulations comprising the agent and uses of the agent.

### BACKGROUND TO THE INVENTION

Cytotoxic compounds are known to be useful both in vivo and in vitro for the treatment, reduction and/or elimination of infections, tumours, cancers, biofilms, plaques, viruses and fungi.

There are many types of cytotoxic compounds having different modes of action, including but not limited to alkylating agents, antibiotics, antimetabolites, free radical generators and mitotic inhibitors. The purpose of all of these effects is the death of unwanted or harmful cells. This purpose can be applied to in vivo and in vitro situations. Compounds that give rise to these effects can vary in form; they may be organic compounds, inorganic molecules, organometallic compounds, metals, salts, gases, biological agents or nanoparticles. It is also known that non-chemical methods can be used to cause cell death, including heat/cold, UV, surgery, radiotherapy and laser therapy.

Cancerous tumours are often treated with combinations of radiotherapy, surgery, laser treatments and chemotherapy. These treatments are often invasive and can cause damage to other cells in the body. Laser treatments can result in excess heat that spreads from the targeted area and harms surrounding cells. Additionally, laser treatments incur high costs due to the use of expensive equipment, as well as often requiring follow-up treatments due to limited effectiveness. Surgery can be imprecise and requires physical entry to where the tumour is located thus limiting its use for some tumours. Chemotherapy exposes the whole body to a toxic chemical, killing many healthy cells as well as tumour cells, and is often associated with unwanted side effects.

Methods of treatment for cancers that cause less damage to healthy cells in the patient, are less invasive and require fewer follow-up sessions would be preferred to the current solutions. The same considerations apply for the treatment of benign tumours such as but not limited to warts, HPV lesions or lipomas. In particular, when growths are in sensitive areas, including genital regions, existing localized treatments with toxic chemicals are flawed due to the severe discomfort to the patient, to the point of intolerability.

The current invention has been devised with this in mind.

### SUMMARY OF THE INVENTION

According to a first aspect of the present invention, there is provided an agent comprising a metal cluster and a molecule comprising a photocleavable group, wherein the molecule forms a structure which at least partially encloses the metal cluster so as to prevent the metal cluster from interacting with a surrounding environment and wherein, upon exposure to light, cleavage of the photocleavable group changes the structure of the molecule such that the metal cluster is able to interact with the surrounding environment.

The invention thus provides a cytotoxic agent that is activatable upon exposure to light. The agent comprises a metal cluster that is at least partially enclosed (i.e. surrounded or protected) by a molecule comprising a photocleavable group. It will therefore be understood that the metal cluster may be partially or completely surrounded or encapsulated by the structure, such that the metal cluster is prevented from interacting with its surroundings (e.g. the surrounding environment, such as surrounding cells or tissues, or components within a cell). This enables the agent to be non-toxic to living cells and organisms, prior to light activation. Upon exposure to light, cleavage of the photocleavable group causes a conformational change in the structure of the molecule, such that the metal cluster becomes exposed to or released into to its local environment. The metal cluster is then able to interact with surrounding cells, tissues or components within cells, including DNA or RNA in the nucleus, cytoplasm, ribosomes or mitochondria. This can result in cell death.

Typically, the agent is capable of crossing cell membranes. The agent may be capable of crossing the nuclear membrane. Activating the agent within a cell provides maximum effectiveness, by targeting the individual cell the agent is in.

In some embodiments the molecule comprises an oligonucleotide. In some embodiments the oligonucleotide comprises DNA, RNA, a synthetic oligonucleotide, or a mixture thereof. In some embodiments the oligonucleotide is DNA.

In some embodiments the structure has the shape of a hairpin comprising a loop (e.g. a single stranded loop) and a double stranded stem.

In some embodiments the double stranded stem comprises one or more G-C base pair(s), such as two or three G-C base pairs. The number and/or position of G-C base pair(s) in the double stranded stem may be varied to change the thermal stability of the structure. In some embodiments the one or more G-C pair(s) are located at or towards the junction of the loop and the double stranded stem. For example, the second and third base pairs of the double stranded stem (the first base pair being adjacent the loop) may be G-C base pairs. This adds stability to the structure.

In some embodiments, the double stranded stem has a GC content of from 10% to 80%, from 20% to 60%, from 25% to 50%, or from 30% to 40%.

In some embodiments the double stranded stem has a length of at least 6 or at least 7 base pairs. In some embodiments the double stranded stem has a length of no more than 15 base pairs, or no more than 12 base pairs. In a preferred embodiment the double stranded stem has a length of 8 or 9 base pairs. A double stranded stem of 8 or 9 base pairs has been found to be optimal for stability.

In some embodiments the loop comprises from 8 to 12 nucleotides (e.g. 8, 9, 10, 11 or 12 nucleotides). In a preferred embodiment the loop comprises 9 nucleotides. The size of the loop limits the size of the metal cluster and therefore affects the toxic effect. A shorter loop advantageously provides stability to the structure, by limiting sensitivity to nuclease activity and by limiting thermal strain on the structure.

At least 50%, 60%, 70%, 80% or 90% of the nucleotides forming the loop may be cytosine. In some embodiments all of the nucleotides of the loop are cytosine. Advantageously cytosine has a strong interaction with the metal cluster which provides effective stabilisation.

In some embodiments the photocleavable group is cleavable by light having a wavelength of from 315 nm to 1 mm, e.g. from 315 nm to 2000 nm In some embodiments the photocleavable group is cleavable by visible light (i.e. light having a wavelength of from 400 to 700 nm). In some embodiments the photocleavable group is cleavable by infrared light (i.e. light having a wavelength of from 700 nm to 1 mm e.g. from 700 nm to 2000 nm). In some embodiments the photocleavable group is cleavable by UV light, e.g. UVA light (i.e. light having a wavelength of from 315 nm to 400 nm). In some embodiments the photocleavable group is cleavable by light having a wavelength of from 350 to 450 nm, or from 380 to 420 nm, e.g. about 405 nm.

In some embodiments the photocleavable group comprises an aminotag phosphoramidite.

In some embodiments, the photocleavable group comprises the following structure:

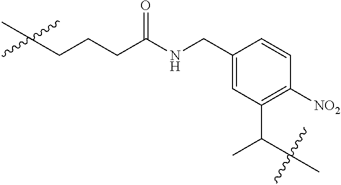 [SMILES: *CCCC(=O)NCc1ccc([N+](=O)[O-])c(C(*)C)c1]

In some embodiments the photocleavable group is located at or towards an end of the loop. Positioning the photocleavable group at or towards an end of the loop (as opposed to the middle of the loop) advantageously leads to increased exposure of the metal cluster upon cleavage, resulting in higher toxicity. In some embodiments the photocleavable group is located at or towards the 5′ end of the oligonucleotide loop.

In some embodiments the metal cluster comprises one or more (e.g. two, three, four or more) metals selected from rhenium, copper, ruthenium, rhodium, palladium, silver, osmium, iridium, platinum, and gold. In some embodiments the metal cluster comprises silver. In some embodiments the metal cluster comprises gold. In some embodiments the metal cluster comprises silver and gold. In some embodiments the metal cluster consists of silver.

In some embodiments the metal cluster comprises no more than 20 atoms, no more than 15 atoms, no more than 10 atoms, no more than 7 atoms, or no more than 5 atoms. It will be appreciated that, within a plurality or a population of agents according to the invention, there may be a range of metal cluster sizes. In such a plurality or a population of agents, the average (i.e. mean) number of atoms in the metal cluster may be from 4 to 11 atoms, or from 5 to 10 atoms, e.g. 7 atoms.

The agent of the invention can be synthesised using standard techniques which are known to those skilled in the art, such as the methods described herein. In some embodiments the agent is synthesised and/or handled in darkened conditions (e.g. in dark rooms and/or using black consumables) to prevent degradation of the agent.

According to a second aspect of the invention there is provided a formulation comprising the agent of the first aspect of the invention and a carrier.

The carrier may be any suitable carrier, such as an aqueous liquid. In some embodiments the carrier is a pharmaceutically acceptable carrier.

In some embodiments the formulation may further comprise one or more components such as a buffer, a stabiliser, and/or an excipient.

According to a third aspect of the invention there is provided the agent of the first aspect, or the formulation of the second aspect, for use as a medicament.

According to a fourth aspect of the invention, there is provided the agent of the first aspect, or the formulation of the second aspect, for use in the treatment of a proliferative disease or condition, or a microbial growth or infection.

The proliferative disease or condition may be cancer or it may be a benign condition.

Wherein the proliferative disease or condition is cancer, in embodiments the cancer may be any of but not limited to carcinoma, sarcoma, lymphoma, brain cancer, blood cancer (e.g. leukaemia, myeloma, myelodysplastic syndromes, myeloproliferative neoplasms), ocular cancer, liver cancer, lung cancer, skin cancer, spleen cancer, colon cancer, intestinal cancer, breast cancer, prostate cancer, testicular cancer, cervical cancer, stomach cancer, kidney cancer, thyroid cancer, ovarian cancer, uterine cancer, oral cancer, oesophageal cancer or pancreatic cancer.

Wherein the proliferative disease or condition is a benign condition, the condition may be any of but not limited to lipoma, a mole, a wart, a lesion (e.g. a HPV-related lesion), angiolipoma, fibroma, benign fibrous histocytomas, hemangioma, benign proliferative breast disease (e.g. atypical ductal hyperplasia, atypical lobular hyperplasia, intraductal papilloma, fibroadenoma), a cyst, or fibrosis.

The microbial growth or infection may be bacterial or fungal. In some embodiments, the microbial growth is a biofilm (e.g. a dental biofilm). The infection may be associated with an abscess or lesion. Advantageously, the agent of the invention can be used to treat infections which are hard to reach (e.g. biofilms) and microbes that are resistant to antibiotics.

In some embodiments the use of the agent or the formulation comprises
(i) administering the agent or the formulation to a subject; and
(ii) exposing the subject to light having a wavelength capable of cleaving the photocleavable group

In some embodiments the subject is an animal. The animal may be a mammal, a fish, an amphibian, a reptiles, or a bird. The mammal may be a human, a rat, a dog, a cat, a horse, a cow, a pig, a mouse, a sheep, a rabbit or a primate. In some embodiments, the subject is human.

In some embodiments the step of exposing the subject to light is carried out a predetermined period of time after the step of administering the agent or the formulation. In some embodiments the step of exposing the subject to light is carried out no more than 90 minutes, no more than 60 minutes, no more than 45 minutes, no more than 30 minutes, no more than 15 minutes, no more than 10 minutes or no more than 5 minutes after administering the agent or the formulation to the subject. Without being bound by theory, it is thought that the agent may be removed from cells via the natural processes by which cells dispose of foreign material. It may be that the agent is only effective when it penetrates the cell nucleus. Therefore, in order to maximise the toxic effect of the agent, it is preferable to expose the subject to light before the agent is removed from the cells.

Alternatively the subject may be exposed to the light immediately after the administration of the agent.

The light may be provided by any suitable light source. In some embodiments the light source is a lamp. In some embodiments the light source is a laser. In some embodiments the light source is natural light.

Prior to administration to the subject, the agent may be pre-treated with a transfection agent (e.g. lipofectamine). This pre-treatment facilitates transfer of the agent through cell membranes.

In some embodiments, the agent is administered at a dose of no less than 2 μM, no less than 5 μM, no less than 10 μM, no less than 15 μM, no less than 20 μM, no less than 30 μM, no less than 40 μM, no less than 50 μM or no less than 100 μM. In some embodiments the agent is administered at a dose of no less than 10 μM. A skilled person will be capable of determining an appropriate dose taking into account relevant factors, such as the type and quantity of cells or tissue being treated.

In some embodiments the agent or the formulation is administered by injection. In some embodiments the agent or the formulation is administered topically.

According to a further aspect of the invention there is provided an in vitro method for the reduction and/or prevention of microbial growth, the method comprising:
(i) contacting the agent or the formulation with a microbial growth or a surface; and
(ii) exposing the microbial growth or the surface to light having a wavelength capable of cleaving the photocleavable group.

In some embodiments the step of exposing the microbial growth or surface to light is carried out no more than 60 minutes after contacting the agent or the formulation to the microbial growth or surface. In some embodiments the light source used is a lamp. In some embodiments the light source used is a laser. In some embodiments the light source used is natural light.

The light may have a power of less than 10 mW, less than 5 mW, less than 2 mW, or less than 1 mW.

In some embodiments, prior to contacting the agent or formulation with the microbial growth or surface (step (i)), the agent or formulation is pre-treated with a transfection agent (e.g. lipofectamine).

In some embodiments the step of exposing the microbial growth or the surface to light is carried out at a predetermined period of time after the step of contacting the agent or the formulation with the microbial growth or surface.

In some embodiments the predetermined period of time is no more than 90 minutes, no more than 60 minutes, no more than 45 minutes, no more than 30 minutes, no more than 15 minutes, no more than 10 minutes or no more than 5 minutes. In some embodiments the microbial growth or surface is exposed to the light immediately after the contacting step.

According to a further aspect of the invention there is provided a method of pre-treating the agent of the first aspect of the invention with a transfection reagent.

In embodiments pre-treating the agent comprises treating the agent with:
(a) a precomplexing reagent (e.g. PLUS reagent (Invitrogen)), optionally at an agent to precomplexing reagent ratio of from 30:1 to 70:1 (e.g. 50:1); and
(b) a transfection reagent (e.g. lipofectamine), optionally at an agent to transfection reagent ratio from 10:1 to 30:1 (e.g. 30:1).

In some embodiments there is an incubation time after treatment according to step (a), and before treatment according to step (b). The incubation time may be more than 5 minutes, e.g. at least 10 or 15 minutes. In some embodiments there is a second incubation time after treatment according to step (b). The second incubation time may be no more than 25 minutes, no more than 20 minutes, no more than 15 minutes or no more than 10 minutes.

In some embodiments step (a) and/or step (b) may be carried out in a suitable buffer, such as ammonium acetate. In some embodiments, step (a) and/or step (b) is not carried out in a cell culture medium. It has been found that the use of a buffer such as ammonium acetate advantageously ensures stability of the agent.

It will be understood that any of the aspects and embodiments described herein may be combined with each other in any combination, unless otherwise stated.

### BRIEF DESCRIPTION OF FIGURES

Embodiments of the invention will now be described by way of example with reference to the figures in which:

FIG. 1 is a schematic diagram of an agent referred to herein as “9C-PC”, according to an embodiment of the invention;

FIG. 2 shows the cell viabilities of treated and untreated cells with 9C-PC where the cleavable group position on the loop is varied;

FIG. 3 shows the optimized emission spectrum of the 9C-PC structure, using a 570 nm excitation;

FIG. 4 shows the maximum fluorescence emission intensity and emission spectral widths for varying stem lengths of hairpins;

FIG. 5 are microscopy images showing the uptake of the 9C-PC agent, modified by inclusion of a Cy5 dye, into mda-mb-231 cells;

FIG. 6 shows Emission intensities for cells treated with 9C-PC (lipofectamine treated [black line]) and 9C-PC (untreated [grey line]);

FIG. 7 shows epidermis tissues treated with various concentrations of 9C-PC, incubated for 30 minutes, and then exposed to a UVA lamp for 30 minutes;

FIG. 8 shows epidermis tissues treated with two concentrations of 9C-PC, incubated for 30 or 60 minutes, and then exposed to a UVA lamp for 30 minutes;

FIG. 9 shows an individual cell before and after treatment with Ag-DNA; and

FIG. 10 shows an area of treated cells within a targeted area.

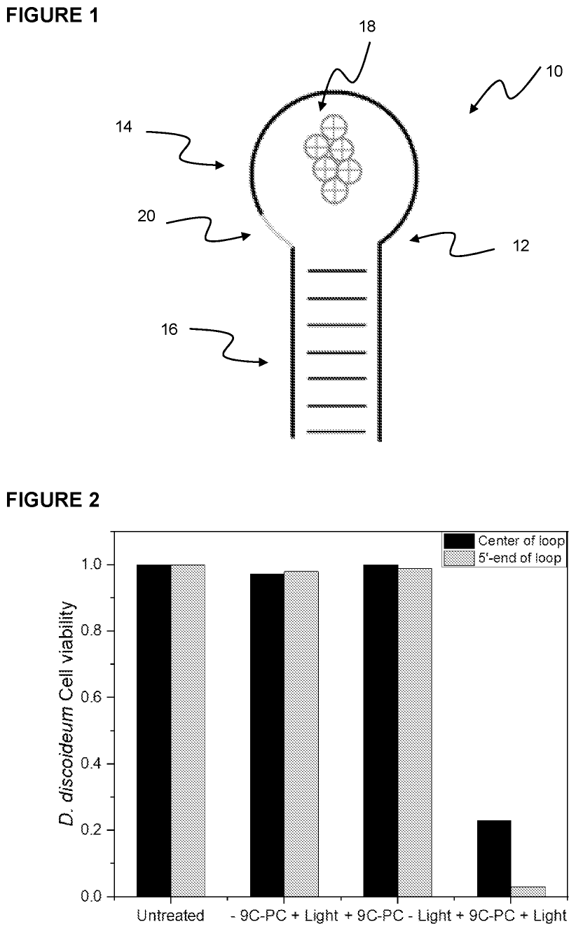

With reference to FIG. 1, the image shows one embodiment of an agent of the invention in a schematic form. The agent 10 comprises a DNA scaffold 12 built out of a synthesized oligonucleotide, forming a hairpin structure (comprising a single-stranded loop 14 and a double-stranded stem 16. The hairpin stabilizes and protects the silver atom cluster (18), of around 10 atoms in size, in the loop 14 of the hairpin. The structure includes a photo-cleavable molecule 20 at the end of the loop (14), that can be cleaved with light having a wavelength of 405 nm photons, thereby opening the loop 14 of the hairpin so as to release the silver cluster 18.

The invention provides a combined solution of chemicals and light which enables the precise targeting and removal of cells, such as removal of cancer cells in a clinical setting. Unlike more aggressive chemotherapy, the agent of the invention is not significantly toxic to the surrounding tissue. The agent is also small enough (only several nanometers) to spread throughout the tissue upon injection, and is able to enter cells and nuclei.

In certain embodiments, the agent can be activated by a weak visible, UV, or IR light beam that will not harm the tissue directly. Upon activation, the agent releases toxic particles in the form of metal (e.g. silver) clusters (preferably <20 atoms in size). These metal clusters are capable of ‘glueing’ nucleic acid molecules together in unusual ways, inhibiting primary cell functions such as transcription of RNAs in the nucleus, to protein synthesis and potentially mitochondrial activity.

Cell death can therefore be stimulated within a designated area, with single cell precision and a potential 100% death rate within the region. The agent of the invention offers a potential improvement on existing methods, and able to induce a slow gradual death of the tissue over the course of several hours. The slow tissue death has the potential to limit formation of scar tissue during the healing process. It therefore provides a less invasive treatment option than existing techniques at relatively low cost.

### EXAMPLES

### Materials and Methods

### Production

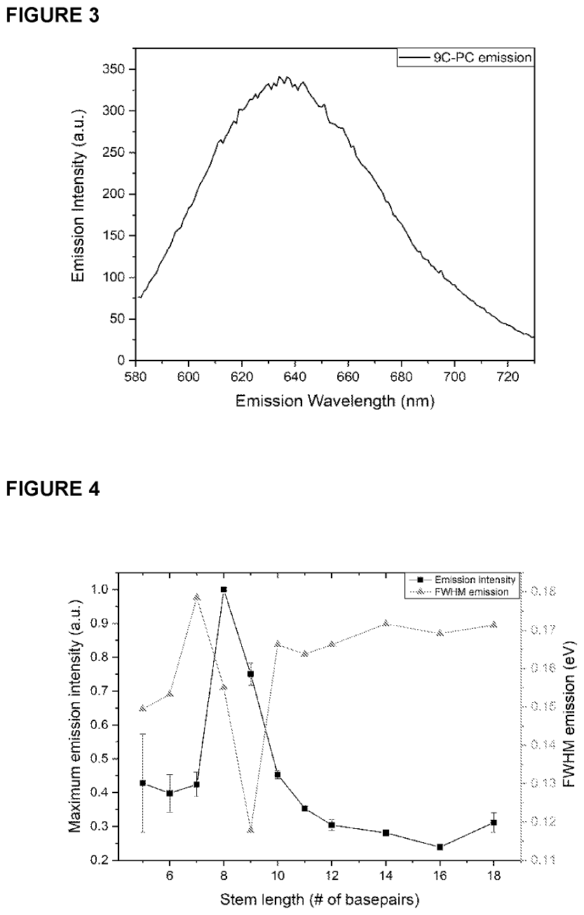

DNA strands (9C-PC sequence 5′-ACTTACCT-(PC)-CCCCCCCCCAGGTAAGT-3′ (SEQ ID NO: 1)) were purchased from Integrated DNA Technologies and used without further purification. A Cy5 fluorescent dye was included on the 5′ end of the hairpin when a fluorescent marker is necessary (FIG. 3). “9C-PC” thus refers to an agent having a loop of 9 cytosine residues and a photocleavable aminotag phosphoramidite group comprising the following structure:

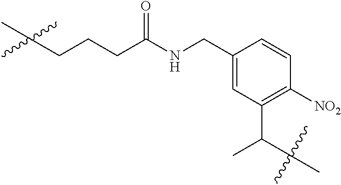 [SMILES: *CCCC(=O)NCc1ccc([N+](=O)[O-])c(C(*)C)c1]

The modified DNA sequence is dissolved in ammonium acetate buffer and exposed to silver ions in the form of silver nitrate, incubated for 30 mins at 4° C., and sodium borohydride is added as a reducing agent to form the metal cluster. The agent is then incubated for 18 hours at 4° C. All steps of the reaction are performed in the dark where possible.

The final concentrations of modified DNA molecules is 10 μM, in a 4 mM ammonium acetate buffer. The Ag⁺:DNA ratio used for synthesis is 7, and the NaBH₄:DNA ratio is 2. The agent is produced in a volume of 1.5 mL.

Before use, the agent is filtrated to remove excess silver and concentrate the sample. First, the sample is concentrated to a volume of 500 μL using 3K Amicon Ultra-0.5 centrifugal filters (Merck Millipore), keeping the volume above 100 μL, to avoid overconcentrating the DNA (13.000 RPM, approx. 7 minutes per cycle). Subsequently, the sample is washed in fresh buffer using the same method three times and concentrated to a volume of 250 μL (to reach approx. 60 μM concentration of the 9C-PC agent).

After filtration, the samples are used immediately or frozen at for later use.

### Application

To help facilitate transport into human cells, the agent is treated with lipid reagents Lipofectamine LTX and PLUS reagent (Invitrogen) before use. The protocol is modified due to the different than standard application:
1) Mix 250 μL 9C-PC+5 μL PLUS Reagent, mix thoroughly, leave 10 mins at approx. 20° C.
2) Add 12.5 μL Lipofectamine, mix gently, leave undisturbed for 20 mins at approx. 20° C.

Injection into the target material is performed by pipetting into the surrounding medium to the desired concentration, followed by 30 mins of incubation. Afterwards, target cells are illuminated with a UVA source (for tissue models or bulk experiments) or a 405 nm laser (for cell culture). The allowed strength of the illumination is determined experimentally through control experiments with identical samples without the 9C-PC agent.

### Microscopy

Imaging of cells incubated with the 9C-PC agent was performed by confocal microscopy set-up equipped with a Yokogawa 10,000 rpm spinning disc unit (Andor Technology Ltd.), Eclipse TiE Nikon inverted microscope (Nikon Corporation) and an IXON Ultra EMCCD camera (Andor Technology Ltd.). A 640 nm laser was used to excite the Cy5 dye for uptake measurements.

### Fluorimetry

To optimize the 9C-PC structure, the excitation and emission properties of the silver clusters were determined using a Cary Eclipse fluorimeter (Varian Medical Systems).

### Cell Culture and Tissue Models

For initial toxicity experiments, Dictyostelium discoideum Wildtype in AX2 cells were cultured in HL5-C medium adjusted to pH 6.7 at 21° C. For microscopy experiments, the cells were harvested below a confluency of 40% and washed three times with PB by centrifugation at 400 g for 5 min. mda-mb-231 cells (human breast cancer) were cultured in Leibovitz's L-15 medium supplemented with fetal bovine serum, 10%. Cell confluency was kept below 50% during culture. For experiments, cells were transferred to 8-well slides (‘μ-slide’, tissue culture treated, Ibidi).

Epidermis tissue models were supplied by the Skin Disease Models group of Leiden University Medical Center, using a confidential procedure. Approx. 1 cm diameter pieces of tissue were grown on plastic filters, after which these were cut into two halves for experiments, whereby one half was exposed to the agent but not the light, as a control experiment (top row in FIG. 6). Tissue models were suspended in medium loaded with the 9C-PC agent for 30 mins before illumination with a UVA lamp for 30 mins. Models were fixed 24 hours after illumination, and cross sections were made for imaging.

### Results

### Positioning of the Photo-Cleavable Group

The photo-cleavable group is positioned such that exposure of the silver is maximized upon excitation with the light. This was tested by utilizing the agent on D. discoideum cells with the cleavable group in two different positions, the centre of the loop and at the base of the loop (i.e. at the 5′ end of the 9C loop).

With reference to FIG. 2, it can be seen that cell viability is not significantly reduced by the presence of light alone or the 9C-PC alone. The measured toxicity of the 9C-PC agent for two different locations of the photo-cleavable spacer is shown, toxicity was determined by cell counting under the microscope. D. discoideum cells were exposed to 5 μM of the agent and exposed to UVA light for 20 mins after a 30 min incubation. Positioning the photocleavable group at the base of the loop shows almost complete effectiveness in inducing toxicity, whereas positioning it in the middle was less effective.

### Optimization of the Length of the Stem

The silver cluster within the 9C-PC agent exhibits a distinct fluorescence. The optical properties of the metal cluster are largely determined by the geometric properties of the cluster within the DNA loop. We therefore used the variance in the optical properties to determine the monodispersity and stability of the Ag-DNA structure. It was observed that for a stem length of 8 or 9 base pairs, fluctuations within the optical properties were minimal, as shown through a stronger and narrower fluorescence peak.

FIG. 4 shows the maximum fluorescence emission from the silver cluster (black squares) occurs and the emission spectral width (grey triangles) as a function of the length of the hybridized part (‘the stem’). A significant maximum in intensity and minimum in spectral width is observed for stems 8 or 9 base pairs in length. Given that the optical properties of the metal cluster depend largely on geometric properties, it can be inferred that the structure is most monodisperse at a length of 8-9 base pairs. The optimized emission spectrum of the 9C-PC structure is shown in FIG. 3, using a 570 nm excitation.

Lipid Reagents for Transport into Human Cells

To facilitate uptake into human cells, lipid reagents can be bound to the 9C-PC structure. This is useful due to the negatively charged DNA double helix being unable to easily transport through the cell membrane. This procedure is well known and used, for example, for transfection of larger DNA fragments. The standard chemical reaction is modified to be more suitable for the 9C-PC agent.

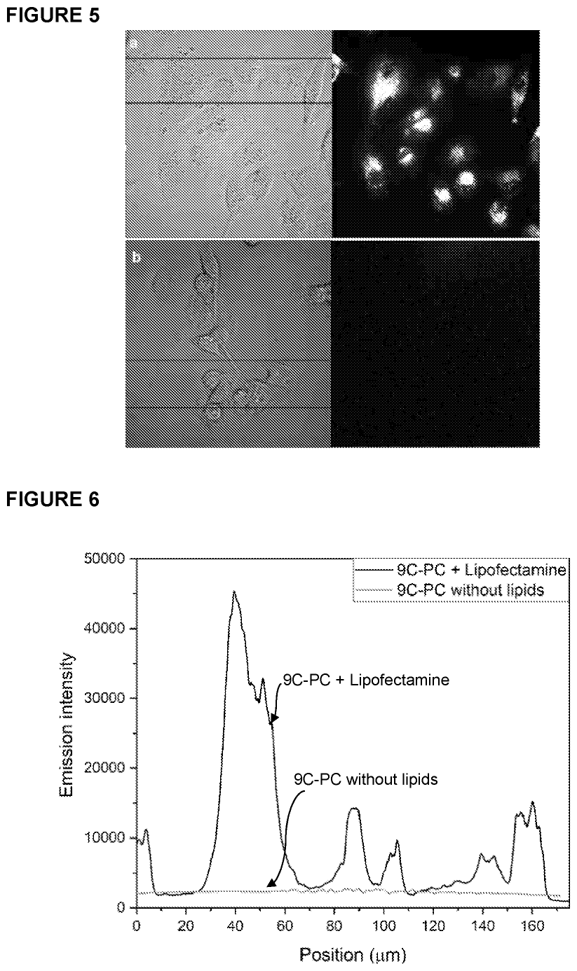

To measure uptake, the 9C-PC agent has a Cy5 fluorescent dye included to track its position in cell culture. The amount of uptake into the cells is measured through the Cy5 fluorescence coming from the cells (FIG. 5). FIG. 5 shows the uptake of the 9C-PC agent, modified by inclusion of a Cy5 dye, into mda-mb-231 cells. Microscopy images are shown for cells after the addition of the agent into the medium, incubation for 20 hours, and replacement of the medium by washing. The top images labelled (a) show microscopy images of cells (left) and fluorescence images (right) after incubation with Cy5 dye-labelled 9C-PC. Lipofectamine is used in a custom synthesis procedure to facilitate the transport of the agent, (b) shows a lack of significant transport into cells without the use of the lipid reagents.

With reference to FIG. 6, the graph shows the profile of emission from the dye attached to the 9C-PC agent. Emission is collected from both images between the black lines. Peaks correspond to significant emission from inside the cells, and therefore exhibit uptake of the agent.

### Tissue Models

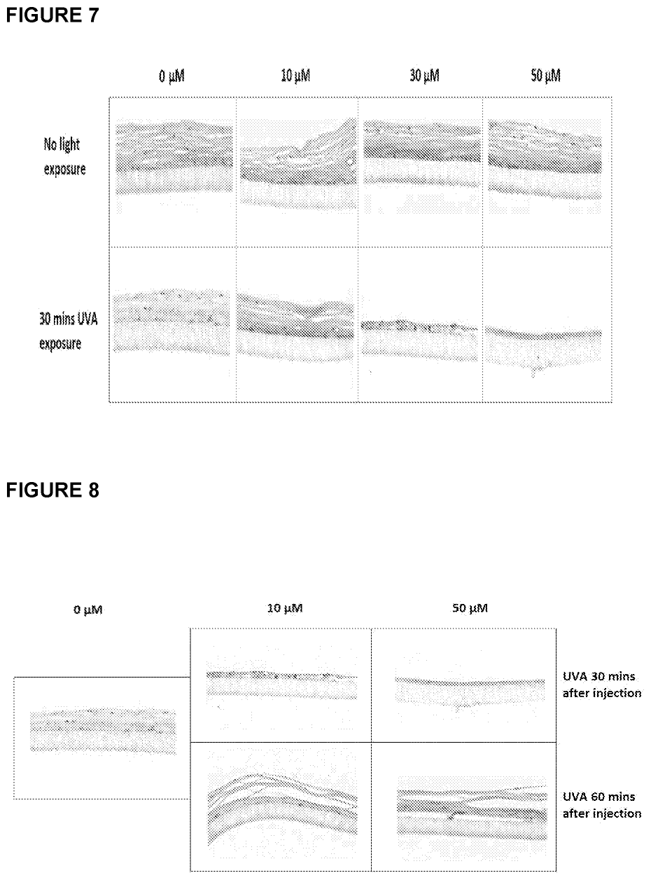

With reference to FIG. 7, the images show microscopy images of cross sections of epidermis tissue models, treated with 0, 10 μM, 30 μM and 50 μM of the 9C-PC agent (left to right). The agent was introduced to the medium and the tissues were exposed through a UVA lamp for 30 mins after a 30 mins incubation. The viability of the tissues can be observed through the remaining thickness of the tissue model, and the appearance of live cells. At 50 μM the tissue appears dead.

A significant reduction of the thickness of the model was observed for concentrations of the 9C-PC agent above 30 μM. A comparison is made between tissues treated with only the chemical agent as a control (top) and when including the light activation (bottom).

### Optimization of Incubation Time Between Application of Agent and Light Exposure

Observations were made on the effectiveness of the agent after 30 mins and 60 mins of incubation, prior to exposure to the light.

FIG. 8 shows microscopy images of cross sections of epidermis tissue models, treated with 0, 10 μM and 50 μM of the 9C-PC agent (left to right). The agent was introduced to the medium and the tissues were exposed through a UVA lamp for 30 mins after a 30 mins (top) or 60 mins (bottom) incubation. The viability of the tissues can be observed through the remaining thickness of the tissue model, and the appearance of live cells. It was observed that incubation for 60 mins with the agent did not increase the effectiveness when activated. This suggests that exposure to light could be performed around 30 mins, preferably no later than 60 mins.

### Use of the Agent in a Human Cancer Cell Culture

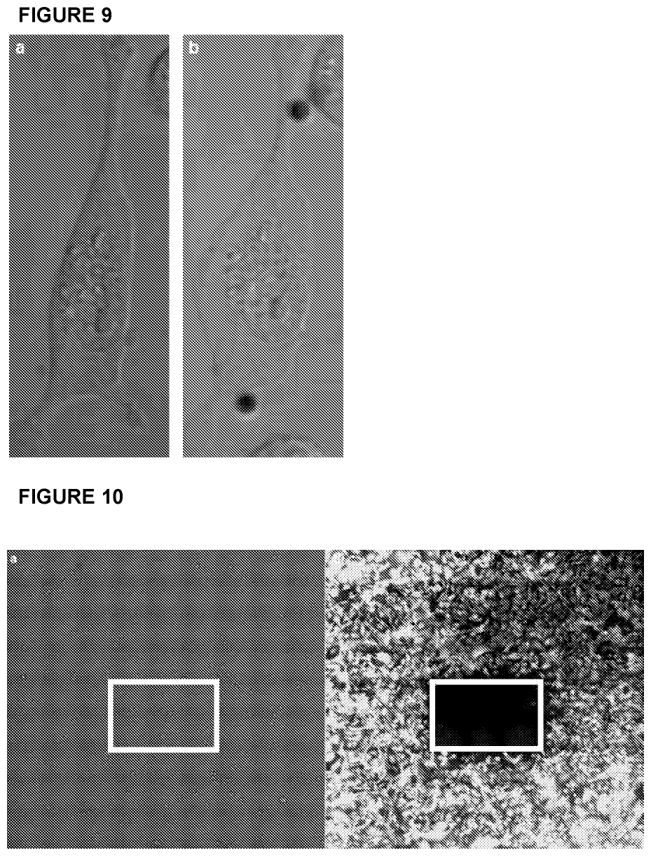

With reference to FIGS. 9 and 10, the images show the functioning of the agent in human cancer cell cultures, using cell line mda-mb-231 (human mammary ductal adenocarcinoma). After the agent was introduced to the environment, exposure to 405 nm light was performed with a scanning laser. Over the course of 2 hours (FIGS. 9a to 9b) the cells die, as exhibited by the loss of shape and lack of mobility within the cell.

Cells were grown densely to cover a glass surface with at least one full layer of cells. After the agent was introduced into the environment, exposure with a 405 nm laser light was performed within the region indicated by the box in FIG. 10a.

Cell death occurs gradually within the region over the course of 2 hours. A cell viability stain (FIG. 10 (b)) was used to exhibit the contrast more clearly. Fluorescein diacetate was used so that green fluorescent light indicates living cells. The black region shows close to 100% effectiveness of the agent, with a precision close to individual cells. The agent shows no significant residual toxicity to surrounding tissue without light exposure.

1. An agent comprising a metal cluster and a molecule comprising a photocleavable group, wherein the molecule forms a structure which at least partially encloses the metal cluster so as to prevent the metal cluster from interacting with a surrounding environment and wherein, upon exposure to light, cleavage of the photocleavable group changes the structure of the molecule such that the metal cluster is able to interact with the surrounding environment.

2. The agent of claim 1, wherein the molecule comprises an oligonucleotide.

3. The agent of claim 1 or claim 2, wherein the structure is a hairpin comprising a loop and a double stranded stem.

4. The agent of claim 3 when dependent on claim 2, wherein the loop comprises from 7 to 12 nucleotides, optionally 9 nucleotides.

5. The agent of claim 3 or claim 4 when dependent on claim 2, wherein the nucleotides of the loop are all cytosine.

6. The agent of any one of claims 3 to 5, wherein the double stranded stem has a length of at least 6 base pairs, optionally 8 or 9 base pairs.

7. The agent of any one of claims 2 to 6, wherein the photocleavable group is located at or towards an end of the loop.

8. The agent of any preceding claim, wherein the photocleavable group is cleavable by light having a wavelength of from 315 nm to 2000 nm.

9. The agent of any preceding claim, wherein the metal cluster comprises or consists of silver.

10. The agent of any preceding claim, wherein the metal cluster comprises no more than 20 atoms.

11. A formulation comprising an agent according to any one of claims 1 to 10, and a carrier.

12. The agent of any one of claims 1 to 10, or the formulation of claim 11, for use as a medicament.

13. The agent of any one of claims 1 to 10, or the formulation of claim 11, for use in the treatment of a proliferative disease or a microbial growth or infection.

14. The agent or formulation for use according to claim 13, wherein said use comprises:
(i) administering the agent or the formulation to a subject; and
(ii) exposing the subject to light having a wavelength capable of cleaving the photocleavable group.

15. The agent or formulation for use according to claim 14, wherein the step of exposing the subject to light (step (ii)) is carried out no more than 60 minutes after administering the agent or the formulation to the subject (step (i)).

16. The agent or formulation for use according to any one of claims 13 to 15, wherein the agent is administered at a dose of no less than 10 μM.

17. The agent or formulation for use according to any one of claims 13 to 16, wherein the agent or formulation is administered by injection.

18. An in vitro method for the reduction and/or prevention of microbial growth, the method comprising:
(i) contacting the agent of any one of claims 1 to 10, or the formulation of claim 11, with a microbial growth or a surface; and
(ii) exposing the microbial growth or the surface to light having a wavelength capable of cleaving the photocleavable group.

19. The agent or formulation for use according to claim 14 or claim 15, or claim 16 or claim 17 when dependent on claim 14, or the method of claim 18, wherein said use or said method further comprises, prior to step (i), pre-treating the agent with a transfection reagent.

20. The method of claim 19, wherein pre-treating the agent comprises treating the agent with:
(a) a precomplexing reagent, optionally at an agent to precomplexing reagent ratio of 30:1 to 70:1; and
(b) a transfection reagent, optionally at an agent to transfection reagent ratio of 10:1 to 30:1.
